(19) United States
(12) Patent Application Publication
(10) Pub. No.: US 2026/0076998 A1
(43) Pub. Date: Mar. 19, 2026

(54) METHOD FOR OBTAINING AN UMBILICAL CORD EXTRACT

(71) Applicant: LUVIGIX, LLC, SAFETY HARBOR, FL (US)

(72) Inventors: Andres Bernardo CAICEDO PALIZ, Quito (EC); Michelle Amanda GRUNAUER ANDRADE, Quito (EC); Luis Alfonso EGUIGUREN LEÓN, Quito (EC); Radmila Francesca VELARDE, Quito (EC)

(73) Assignee: THE UNIVERSIDAD SAN FRANCISCO DE QUITO USFQ, Quito (EC)

(21) Appl. No.: 18/994,328

(22) PCT Filed: Jul. 7, 2023
(86) PCT No.: PCT/IB2023/057019

§ 371 Date: Dec. 5, 2025

(30) Foreign Application Priority Data
Jul. 14, 2022 (EC) SENADI-2022-48957

(51) Int. Cl. A61K 35/51; A61P 17/02

(52) U.S. Cl. CPC A61K 35/51; A61P 17/02

(57) ABSTRACT

The present invention relates to a method for obtaining an umbilical cord extract that can be used for tissue regeneration or repair, for medical or cosmetic uses, as a preparation base for liquid, semisolid or solid grafts or dressings, binders or polymerising agents, or in the subsequent purification of the regenerative components thereof.

### TECHNICAL FIELD

The present disclosure refers to the general field of medicine, particularly to cell therapy and regenerative medicine, and more particularly to a method for the preparation of an umbilical cord extract that allows maintaining the cell regeneration properties for its application as a base of preparations for liquid, semi-solid or solid dressings or grafts, binders, polymerizers, or in the subsequent purification of their regenerative components and for the stimulation of cellular processes.

### BACKGROUND

Various methodologies have been described to obtain cellular or acellular extracts from the umbilical cord or fractions thereof, due to the multiple applications that such extracts have in regenerative medicine. However, many of these procedures do not guarantee that the extract obtained retains its regenerative properties, generating results that are not very reproducible and do not guarantee the desired effects of its application.

For example, patent US20210369786A1 discloses a method for producing an extract from fetal tissues or a newborn animal, using PBS where the possible starting tissues or organs include musculoskeletal tissue, nervous tissue, adipose tissue, epithelial tissue and cord tissue. umbilical. Patent US20180028569A1 refers to a method for the preparation of a protein extract from umbilical cord that allows obtaining a protein concentration of the soluble component of at least around 0.05 micrograms of total protein extract. Other disclosures are particularly aimed at obtaining viable cell extracts, such as the article by Smith et al (Smith et al. 2016. Standardizing umbilical cord mesenchymal stromal cells for translation to clinical use: Selection of GMP-compliant medium and a simplified isolation method. Stem Cells International; Volume 2016, Article ID 6810980 https://doi.org/10.1155/2016/6810980). The generation of an umbilical cord extract through different processes seeks to take advantage of the regenerative properties of the cells in this tissue through its secretome in terms of stimulating cell proliferation and migration. Disclosures such as those by Bakhtyar et al (Bakhtyar et al. 2017. Acellular Gelatinous Material of Human Umbilical Cord Enhances Wound Healing: A Candidate Remedy for Deficient Wound Healing. Front. Physiol; Volume 8, 2017 https://doi.org/10.3389/fphys.2017.00200), show that said extract promoted cell migration. However, no effects were observed on cell proliferation or viability in fibroblasts. That is to say, the need persists for a procedure that ensures the obtaining of an umbilical cord extract that preserves its cell regeneration properties for its various medical and cosmetic applications.

### SHORT DESCRIPTION

The present disclosure refers to a method for preparing an umbilical cord extract that can be applied in regeneration, tissue repair, in medical or cosmetic applications, as a base for preparations for liquid, semi-solid or solid dressings or grafts, binders, polymerizers, or in the subsequent purification of its regenerative components. The proposed method involves reserving between one and five umbilical cords after birth in a physiological or buffered solution; wash, fractionate and homogenize the cords in buffered cell culture medium; centrifuge the mixture obtained and filter the crude liquid fraction obtained with a clarification filter between 1 μm to 100 μm, followed by a second filtration by pore size for microfiltration between 0.1 μm to 1 μm. In particular, all steps of the method are carried out at a temperature between 1 and 10° C.

### BRIEF DESCRIPTION OF FIGURES

FIG. 1 Sequence of steps of the method to prepare an umbilical cord extract:

A. One to three umbilical cords are placed in a physiological or buffered solution.

B. Wash, fractionate and homogenize the cords in buffered cell culture medium.

C. Centrifuge the mixture obtained in B and filter the crude liquid fraction obtained with a clarification filter between 1 μm to 100 μm, followed by a second filtration by pore size for microfiltration between 0.1 μm to 1 μm

FIG. 2 Effects of umbilical cord extract on the proliferation of human fibroblasts isolated from foreskin. On the Y axis, number of cells (×1000). The bars on the X axis show the different assays, where X in the figure is equivalent to 6 gg/ml. Five trials were performed with three repetitions for each condition. Post hoc Kruskal-Wall is and Mann-Whitney, unpaired, non-parametric tests were performed (*p<0.05, **p<0.01, ***p<0.001).

FIG. 3 Representative images of the effects of umbilical cord extract on the migration of human fibroblasts isolated from foreskin. The closure area analyzed is observed after having cultured the fibroblasts for 16 hours with the extract. The migration front of the cells is observed (in black), from left to right, where when using the extract in concentrations X, X2, X4 there is greater dispersion and closure of the area. Representative images of the wound closure area are shown for each concentration, the field length is 700 μm. The migration of fibroblasts and wound closure is observed from left to right. Condition X in the figure is equivalent to 6 μg/ml.

FIG. 4 Estimated cell migration in-vitro through the “wound healing” assay. The closure area was analyzed after having cultured the fibroblasts for 16 hours with the HUC extract. The measurement of the wound areas was carried out with the ImageJ program. Condition X is equivalent to 6 μg/ml. The size of the area is taken from FIG. 3 (Y axis) and compared between conditions. Five trials were performed with three repetitions for each condition. The program used to perform the statistical analysis was GraphPad prism 6. Statistical analysis: Mean±SD; Post hoc Kruskal-Wallis and Mann-Whitney, unpaired, non-parametric tests were performed (*p<0.05, **p<0.01, <0.001).

FIG. 5 The metabolic activity of fibroblasts that were cultured with the HUC extract for 72 h is shown. Data were collected after measurement with the spectrophotometer at 570 nm. Condition X is equivalent to 6 g/ml. The program used to perform the statistical analysis was GraphPad prism 6. Statistical analysis: Mean±SD; Non-paired, non-parametric Kruskal-Wallis and Mann-Whitney post hoc tests were performed (*p<0.05, **p<0.01, ***p<0.001).

### DETAILED DESCRIPTION

### Method to Prepare an Umbilical Cord Extract

The present disclosure relates to a method for preparing an umbilical cord extract comprising: a—reserving between one and five umbilical cords after birth in a physiological or buffered solution; b—wash, fractionate and homogenize the cords in a buffered cell culture medium; and c—centrifuge the mixture obtained in b—and filter the crude liquid fraction obtained with a clarification filter between 1 μm to 100 μm, followed by a second filtration by pore size for microfiltration between 0.1 μm to 1 μm, where all the steps of the method are carried out at a temperature between 1 and 10° C.

For the purposes of the present disclosure, an umbilical cord extract can be obtained from the umbilical cords of any mammal.

Between one and five umbilical cords are reserved after birth in a physiological or buffered solution at a temperature between 1 and 10° C. In one embodiment, the cords are maintained in a sterile environment throughout the processing, for example, in a laminar flow chamber or any other that guarantees a state of sterility during processing and transportation. The cords should be kept wet in the solution.

For the purposes of the present disclosure, the physiological or buffered solution is a solution of any medium known in the art, appropriate for cell culture. mammals, selected from, but not limited to: MEM (minimum Essential Medium), IMDM Iscove's Modified Dubelco's Medium, RPMI (Roswell Park Memorial Institute medium), HAM's F-10 and F-12, Dublecco's Modified Eagle's Medium DMEM. In one embodiment, the solution may contain antibiotics selected from penicillin at a concentration of 50 to 100 IU/ml, and streptomycin, at a concentration of 50 to 100 μg/mL.

In one embodiment, the cords are washed with a physiological or buffered solution at a temperature between 1 and 10° C., until any remaining blood is completely removed; at least one wash is required. In a particular modality, three washes are carried out.

The washed cords are fractionated and homogenized in the medium in a proportion of 10 g per 100 ml, by any method known in the art, selected from, but not limited to, grinding, homogenization, lysis, sonication. The homogenization or grinding of the tissue can be carried out for 1 minute to 1 hour at speeds from 60, 200 to 20,000 revolutions per minute. In a particular modality, homogenization is carried out for 10 minutes at a temperature between 1 and 10° C. and speed 1600 rpm.

The mixture of the homogenized umbilical cord in the physiological or buffered solution is allowed to rest for 1 minute to 1 h. In a particular modality, the rest time is 5 minutes.

The mixture is centrifuged between 1 min to 1 h, at a force of 50 to 1000×g. In a particular embodiment the mixture is centrifuged for 1 minute at a force of 500×g.

The crude liquid fraction can be preserved at temperatures from −20 to −220° C. In a particular embodiment the crude liquid fraction is preserved at −80° C.

The raw liquid fraction, immediately processed or preserved, is filtered by any clarification means known in the art by pore size between 1 μm to 100 μm. In one embodiment, sterile filter paper is used. In another particular embodiment, Whatman grade 1 filter paper with a pore size of 11 μm is used and the filtration is carried out at a temperature between 1 and 10° C. The filtered solution is taken to a biosafety chamber or sterile processing environment. Microfiltration is performed by any means known in the art, for pore size between 0.1 μm to 1 μm. In a particular embodiment, microfiltration is carried out through a pore size of 0.22 μm, at a temperature between 1 and 10° C.

In a particular embodiment, all steps of the method are carried out at a temperature between 1 and 10° C. In a more particular embodiment, the steps are carried out at a temperature of 4° C. In this way, it is guaranteed that during the isolation, processing and maintenance of the extract, at a low temperature, the subcellular components released maintain their structure, biological properties and replicability in their application. In preliminary tests, it was observed that an increase in temperature of more than 10° C. limits the effect and replicability of the results. Carrying out all stages at a temperature between 1 and 10° C. until application to fibroblasts promotes cell migration and proliferation. Thus, the present method allows the structural stability and biological effects of the extract to be preserved.

Once the extract is obtained, its composition is estimated using any methodology known in the art, selected from, but not limited to, electrophoresis, mass spectrophotometry, spectrophotometry, immunoassays, bioluminescence, detection of nucleic acids, separation by centrifugation and filtration.

In a particular embodiment, the extract obtained contains extracellular vesicles including and not limited to exosomes (30-150 nm); microvesicles (50-500 nm); apoptotic bodies (50-2000 nm), nucleic acids, proteins, lipids, and combinations thereof. In one embodiment, the extract shows a total protein concentration up to approximately 100 μg/ml, quantified through the Bradford method by spectrophotometry. In observations by phase contrast microscopy and with a 100× objective, vesicles of different sizes or subcellular bodies are observed.

In a particular embodiment, the amount of protein in the solution is estimated with any protein quantification method, selected from, but not limited to, Bradford, WesternBlot, Quantitative Proteomic Analysis, fluorescent quantification, colorimetric and the like, being able to find from 1 ng to 100 ug. In a modality in particular, protein quantification is carried out using the Bradford methodology. Extracts can be obtained with a total protein amount of approximately up to 100 μg/ml.

The extract obtained maintains its capacity for cell regeneration as can be observed by proliferation, cell migration and MTT assays (colorimetric assay of cellular metabolic activity, with 3-(4,5-dimethylthiazol-2-yl)-2 bromide, 5-diphenyltetrazol-MTT).

In a particular embodiment, it is observed that the extract increased cell proliferation by 30% when tested on skin fibroblasts in vitro with concentrations between 12.5 to 25 μg/ml. In ‘wound healing’ assays, aimed at estimating cell migration, a wound or space is generated in the cell mat in vitro and the ability of the cells to reduce the area and generate a migration front is observed. Cells exposed to the extract can migrate and reduce space by 10-15%. Through the MTT metabolic activity test, it was observed that cells exposed to concentrations of the extract between 12.5 to 50 μg/ml can increase their metabolic activity by 20 to 30% compared to the control.

### Uses of Umbilical Cord Extract

The umbilical cord extract obtained by the method described in the present disclosure can be used in applications that include, but are not limited to, medical applications intended for the repair and regeneration of tissues and cells, basal media for cell culture, solutions for subcutaneous, intraorgan, blood applications of intravenous, intravascular type, part of immersion solutions, part of serums, liquids, dehydrated, lyophilized, evaporated, sublimated, and as components or cosmetic products and food supplements.

In particular modalities, the extract is used as a base for preparations for liquid, semi-solid or solid dressings or grafts, binders, polymerizers, or in the subsequent purification of its regenerative components. The present invention will be presented in detail through the following examples, which are provided for illustrative purposes only and not with the aim of limiting its scope.

### EXAMPLES

### Example 1

### Method for Preparing an Umbilical Cord Extract

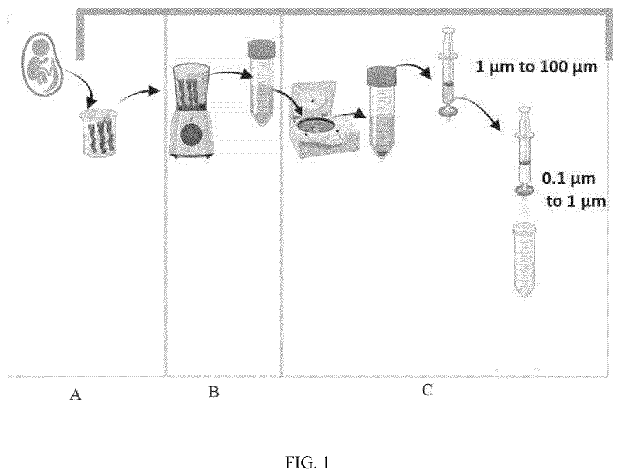

The method for preparing an umbilical cord extract is detailed in FIG. 1.

3 cords were taken immediately after birth, placing them in a physiological buffered saline solution (Phosphate Buffered Saline—PBS) at 4° C., they were washed with a PBS solution at 4° C., in a flow chamber. To remove all traces of blood, three washes were performed.

The cords were cut transversely or longitudinally into small units (0.5 to 10 cm), processed in a food processor with power of 500 W, sufficient power to homogenize the tissue (1600 rpm), for 10 minutes and mixed with Dubelco modified Eagles medium (DMEM), at a temperature of 4° C., in a proportion of cord pieces and enriched medium of 10 g in 100 ml.

The crushed cord mixture was collected and allowed to rest for 5 minutes. It was centrifuged for 1 minute at a force of 500×g. The crude liquid fraction was taken and stored at −80° C. The crude liquid fraction, immediately processed or preserved, was filtered through sterile, grade 1 paper with 11 μm pore size and taken to a biosafety chamber or sterile processing environment. Filtration was performed using a pore size of 0.2 μm and the extract was collected.

The amount of total protein of the extract obtained was determined using the Bradford methodology, which was at an average value of 100 μg/ml.

### Example 2

### Cell Proliferation Activity Assay

On day 0, isolated foreskin fibroblasts were cultured in a T75 culture flask at 80% confluency equivalent to 1 million to 1.5 million cells in 10% DMEM+Fetal Bovine Serum+penicillin streptomycin according to the state of the art. On day 1, 10,000 cells per well are cultured in 10% DMEM+Fetal Bovine Serum. On day 2 it was changed to 1% DMEM medium. On day 3, the cells were washed and exposed to human umbilical cord (HUC) extract obtained by the procedure of Example 1, for 48 h. Subsequently, the cells were detached with 0.25% trypsin-EDTA and counted.

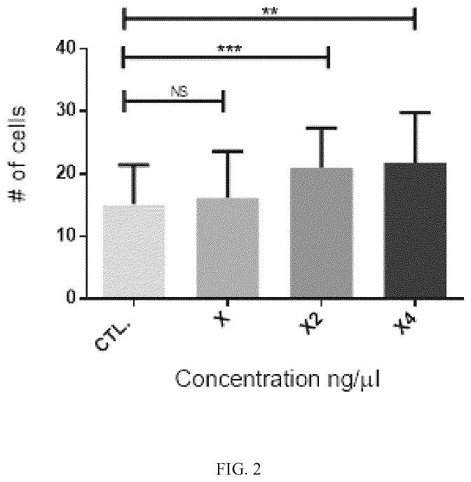

The results are illustrated in FIG. 2. The Five trials were performed with three repetitions for each condition. Non-paired, non-parametric Kruskal-Wallis and Mann-Whitney post hoc tests were performed (*p<0.05, **p<0.01, ***p<0.001).

### Example 3

### “Wound Healing” Cell Migration Activity Assay

Fibroblasts were cultured as described in Example 2 until day 2. On day 3 the cells reached 100% confluence. On day 4 it was changed to 1% DMEM medium. On day 5, the well with confluent cells was wound with a 200 μl pipette tip, washed, and the cells were exposed to HUC extract for 16 h. Subsequently, three representative fields with a length of 700 μm were imaged.

A 10-15% increase in cell migration was estimated at the highest concentration of the extract (X4). Five trials were performed with three repetitions for each condition. Post hoc Kruskal-Wallis and Mann-Whitney, unpaired, non-parametric tests were performed (*p<0.05, **p<0.01, ***p<0.001).

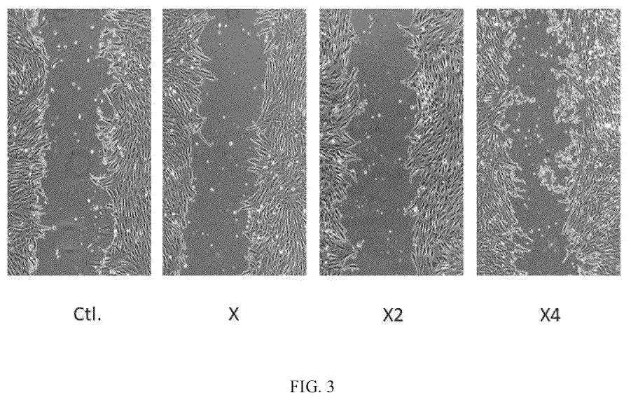

FIG. 3 illustrates the effects of umbilical cord extract on the migration of human fibroblasts isolated from foreskin. The closure area analyzed is observed after having cultured the fibroblasts for 16 hours with the extract. The black line that runs along the migration front is observed. Representative images of the area are shown wound closure for each concentration, the field length is 700 μm. The migration of fibroblasts and wound closure is observed from left to right.

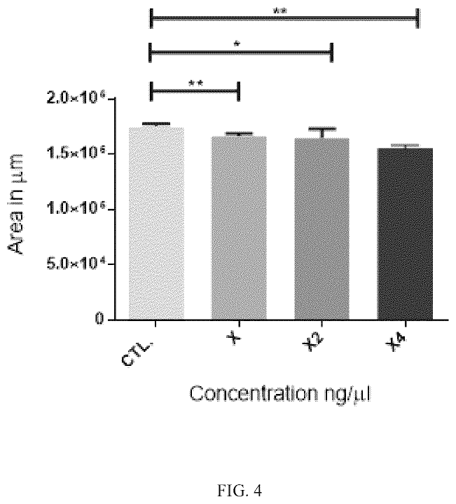

The results illustrated in FIG. 4 correspond to the cell migration estimated in vitro through the “Wound Healing” assay. The closure area was analyzed after having cultured the fibroblasts for 16 hours with the HUC extract. The measurement of the wound areas was carried out with the ImageJ program. Condition X is equivalent to 6 μg/ml. The size of the area is taken from FIG. 3 and is compared between conditions. Five trials were performed with three repetitions for each condition. The program used to perform the statistical analysis was GraphPad prism 6. Statistical analysis: Mean±SD; Post hoc Kruskal-Wallis and Mann-Whitney, unpaired, non-parametric tests were performed (*p<0.05, **p<0.01, ***p<0.001).

### Example 4

### Metabolic Activity Assay by MTT

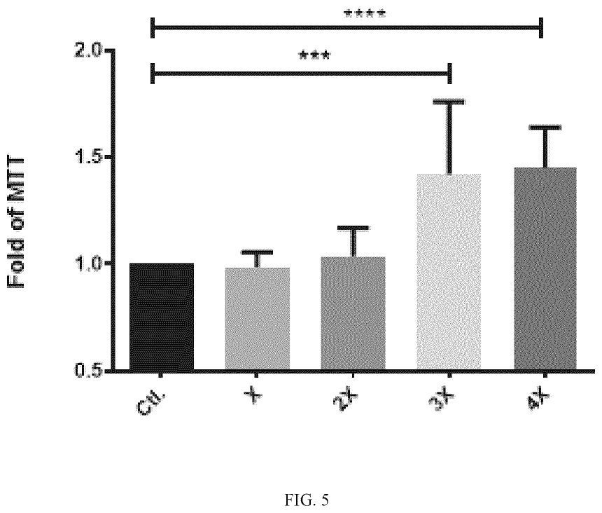

Isolated foreskin fibroblasts were cultured in 10% DMEM+Fetal Bovine Serum at 25,000 cells per well. On day 2, it was washed with physiological solution (PBS) and the cells were exposed to HUC extract for 72 h. Subsequently, 3-(4,5-dimethylthiazol-2-y1)-2,5-diphenyltetrazol bromide (MTT) was added to the medium at a concentration of 500 μg/ml. After 2 h of incubation, it was washed with PBS and 250 μl of DMSO were added. 100 μl of the solution with the MTT diluted in DMSO was taken and added to one well of a 96-well plate for ELISA. The reading was carried out in a spectrophotometer at 570 nm. In the analysis, background noise was removed from the medium, and the data were transformed relative to the control. It was observed that cells exposed to concentrations of the extract between 12.5 to 50 μg/ml increased their metabolic activity between 20 to 30% compared to the control. Five trials were performed with three repetitions for each condition. Non-paired, non-parametric Kruskal-Wallis and Mann-Whitney post hoc tests were performed (*p<0.05, **p<0.01, ***p<0.001). The results are illustrated in FIG. 5.

What is claimed is:

1. A method for preparing an umbilical cord extract comprising the steps of:
a—reserving between one and five umbilical cords after birth in a physiological or buffered solution;
b—wash, fractionate and homogenize the umbilical cords in a buffered cell culture medium; and
c—centrifuge the mixture obtained in b-and filter a crude liquid fraction obtained with a clarification filter between 1 μm to 100 μm, followed by a second filtration by pore size for microfiltration between 0.1 μm to 1 μm, where all the steps of the method are carried out at a temperature between 1 and 10° C.

2. The method of claim 1, wherein in step b—a culture medium selected from Dulbecco's modified Eagles medium (DMEM), minimum essential medium (MEM), Iscove's Modified Dulbecco's Medium (IMDM), Roswell Park Memorial Institute medium (RPMI), or HAM's F-10 and F-12 nutrient mix is used.

3. The method of claim 2 wherein in step b—DMEM medium is used, and the beads are in a proportion of 10 g per 100 ml of medium.

4. The method of claim 1, wherein step c—is carried out through filtration with a syringe and a piston or centrifugation at between 50 to 1000×g, for between 1 to 20 minutes.

5. The method of claim 1, wherein the crude liquid fraction obtained in step c-is filtered through a pore size of 11 μm, followed by a second filtration through a pore size of 0.22 μm.

6. An umbilical cord extract obtained using the method of:
reserving between one and five umbilical cords after birth in a physiological or buffered solution;
b—wash, fractionate and homogenize the cords in a buffered cell culture medium; and
c—centrifuge the mixture obtained in b—and filter the crude liquid fraction obtained with a clarification filter between 1 μm to 100 μm, followed by a second filtration by pore size for microfiltration between 0.1 μm to 1 μm,
wherein all the steps of the method are carried out at a temperature between 1 and 10° C.

7. The umbilical cord extract of claim 6, wherein the umbilical cord extract is used for medical applications.

8. The umbilical cord extract of claim 6, wherein in step b—a culture medium selected from DMEM, MEM, IMDM, RPMI, HAM's F-10 and F-12 is used.

9. The umbilical cord extract of claim 8, wherein in step b—DMEM medium is used, and the beads are in a proportion of 10 g per 100 ml of medium.

10. The umbilical cord extract of claim 6, wherein in step c—it can be carried out through filtration with a syringe and piston or centrifugation at between 50 to 1000×g, for between 1 to 20 minutes.

11. The umbilical cord extract of claim 6, wherein the crude liquid fraction obtained in step c-is filtered through a pore size of 11 μm, followed by a second filtration through a pore size of 0.22 μm.

12. The umbilical cord extract of claim 6, wherein the umbilical cord extract is used for cosmetic purposes.

13. The umbilical cord extract of claim 6, wherein the umbilical cord extract is used for the repair and regeneration of tissues and cells.
